(19) United States
(12) Patent Application Publication
(10) Pub. No.: US 2018/0312458 A1
(43) Pub. Date: Nov. 1, 2018

(54) METHOD F0R THE PURIFICATION OF CYCLOHEXADEC-8-EN-1-ONE

(71) Applicant: BASF SE, Ludwigshafen am Rhein (DE)

(72) Inventors: FRAUKE THRUN, Mannheim (DE); Joaquim Henrique TELES, Waldsee (DE); Albert WERNER, Bishop, TX (US); Ralf PELZER, Fürstenberg (DE)

(21) Appl. No.: 15/766,425

(22) PCT Filed: Oct. 7, 2016
(86) PCT No.: PCT/EP2016/074016

§ 371 Date: Apr. 6, 2018

(30) Foreign Application Priority Data
Oct. 8, 2015 (EP) 15188864.1

(51) Int. Cl. C07C 49/587; C07C 45/28; C07C 45/82; C07C 45/54; C07C 45/79

(52) U.S. Cl. CPC C07C 49/587; C07C 45/28; C07C 2523/72; C07C 45/54; C07C 45/79; C07C 45/82

(57) ABSTRACT

The present invention relates to a novel method for purifying cyclohexadec-8-en-1-one, a method for preparing cyclohexadec-8-en-1-one and cyclopentadecenones, the substances and substance mixtures prepared therefrom and use thereof as aroma substances, particularly as fragrances, and also aroma substance compositions and agents comprising these mixtures.

The present invention relates to a novel method for purifying cyclohexadec-8-en-1-one, a method for preparing cyclohexadec-8-en-1-one and cyclopentadecenones, the substances and substance mixtures prepared therefrom and use thereof as aroma substances, particularly as fragrances, and also aroma substance compositions and agents comprising these mixtures.

### BACKGROUND OF THE INVENTION

In the perfume industry there is a constant demand for new fragrances suitable as fragrance compositions or perfumed articles.

There is a particular demand for musky fragrances and fragrance compositions. This is understood to mean an odor which is similar to the natural musk scent.

Cyclohexadec-8-en-1-one (as a cis/trans isomeric mixture) is a commercial fragrance (Globanone® from Symrise). Trans- and cis-cyclopentadec-8-en-1-one are already mentioned in U.S. Pat. No. 5,936,100 and in Fürstner, A. et al, Synthesis 1997, 792. Starting from heptadec-1,16-dienone, the compounds were prepared by means of a ruthenium-catalyzed ring closing alkene metathesis.

Globanone® is prepared industrially from cyclohexadeca-1,9-diene (CHDD, EP-A-1 288 181) via a two-stage synthesis:

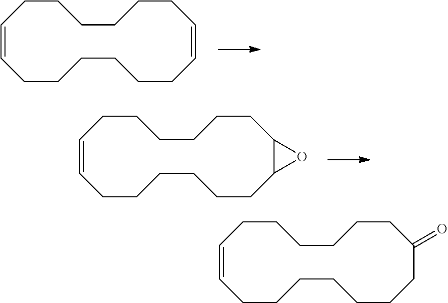

Since both stages are afflicted with problems, there is a desire to develop a simpler one-stage synthesis.

The one-stage oxidation of cyclohexadeca-1,9-diene with N₂O is known from the prior art (WO 2012/084673). The main product formed in this case is (E/Z)-cyclohexadec-8-en-1-one and also various cyclopentadecenyl carbaldehydes as byproducts. Separation by distillation of the mixture of cyclohexadec-8-en-1-one and cyclopentadecenyl carbaldehyde is only possible with a high loss of yield of cyclohexadec-8-en-1-one. The mixture of cyclohexadec-8-en-1-one and cyclopentadecenyl carbaldehyde must however be separated since otherwise cyclohexadec-8-en-1-one is afflicted with an off-note (odor of plastic).

The use of cis- and trans-cyclopentedec-7-en-1-one as additive in a formulation for inhibiting melanin synthesis is mentioned in EP-A-1 264 594. EP-A 216 185 mentions that the cis-7- and 8-cyclopentadecen-1-one compounds can be prepared via an acid or Lewis acid catalysed Meerwein rearrangement, starting from cis-16-oxabicyclo-[13.1.0]-hexadec-8-ene, without however providing any experimental proof thereof. The usability of both compounds as fragrances is also postulated. The actual olfactory properties of the respective (E/Z)-7- and -8-cyclopentadecenones have not, however, been described in the literature to date.

The Cu-catalyzed oxidative decarbonylation is known and has been widely published and examples may be found in: a) Tetrahedron Letters 1969, 12, 985; U.S. Pat. No. 3,496,197; b) 1995 Tetrahedron Letters 4641, c) Org. Lett. 2010, 2630, d) Bioorg. Med. Chem. Lett. 2013, 23, 5949, e) Chin. Chem. Lett. 2014, 25, 771.

The object therefore consisted of finding a method in which the aldehydes, formed as byproducts in the synthesis of Globanone® by N₂O oxidation, can be removed economically. Since separation by distillation alone does not achieve the goal, a chemical separation is preferable. Chemical separations of aldehydic byproducts from ketones prepared by N₂O oxidation are likewise known from the prior art (WO2008/000752). Although these methods, which are based on an aldol condensation, can very effectively convert the aldehyde into readily removable high boilers, this leads to one mole of ketone being sacrificed per mole of aldehyde. The object therefore particularly consisted of finding an improved method which does not lead to ketone losses and generates as far as possible a further product of value from the aldehydes.

### SUMMARY OF THE INVENTION

The above object, surprisingly, was achieved in particular by providing the method for purifying Globanone defined in the claims. The cyclopentadecenyl carbaldehydes are subjected to a Cu-catalyzed oxidative decarbonylation, wherein they are degraded to cyclopentadecenones, which can then be readily separated by distillation from cyclohexadec-8-en-1-one. The cyclopentadecenones themselves are valuable musk fragrances.

### DETAILED DESCRIPTION OF THE INVENTION

### a) General Definitions

If no other details are provided, the chemical formula names used herein include any stereoisomeric form of the compound, and also any mixtures of these stereoisomeric forms.

“Cyclohexadecenone” is, in particular, “cyclohexadec-8-en-1-one”, comprising (E)-cyclohexadec-8-en-1-one and (Z)-cyclohexadec-8-en-1-one in stereoisomerically pure form or as a mixture of E and Z isomers.

“Cyclopentadecenone” comprises cyclopentadec-8-en-1-one and/or cyclopentadec-7-en-1-one and particularly (E)-cyclopentadec-8-en-1-one and/or (E)-cyclopentadec-7-en-1-one and also (Z)-cyclopentadec-8-en-1-one and/or (Z)-cyclopentadec-7-en-1-one, and also stereoisomeric mixtures thereof.

“Cyclopentadecenyl carbaldehyde” comprises cyclopentadec-8-en-1-one and/or cyclopentadec-7-en-1-one and particularly (E)-cyclopentadec-8-enyl-1-carbaldehyde and/or (E)-cyclopentadec-7-enyl-1-carbaldehyde and also (Z)-cyclopentadec-8-enyl-1-carbaldehyde and/or (Z)-cyclopentadec-7-enyl-1-carbaldehyde, and also stereoisomeric mixtures thereof.

“Cyclohexadecadiene” is particularly cyclohexadeca-1,9-diene, in particular the (E/E), (Z/Z), (E/Z) or (Z/E) form of cyclohexadeca-1,9-diene, and also stereoisomeric mixtures thereof.

“Fragrances prepared according to the invention” particularly comprise the above “cyclohexadecenones” and “cyclopentadecenones” and also corresponding substance mixtures thereof.

An “aroma chemical” is a generic term for compounds which may be used as “fragrance” and/or as “flavoring”.

In the context of the present invention, “fragrance” is understood to mean natural or synthetic substances having intrinsic odor.

In the context of the present invention, “flavoring” is understood to mean natural or synthetic substances having intrinsic flavor.

In the context of the present invention, “odor” or “olfactory perception” is the interpretation of the sensory stimuli which are sent from the chemoreceptors in the nose or other olfactory organs to the brain of a living being. The odor can be a result of sensory perception by the nose of fragrances, which occurs during inhalation. In this case, the air serves as odor carrier.

In the context of the present invention, “scent” is understood to mean a pleasant smelling odor. The same applies to an “scent substance” according to the invention.

In the context of the present invention, a “perfume” is a mixture of fragrances and carriers such as, in particular, an alcohol.

In the context of the present invention, a “perfume composition” is a perfume comprising different amounts of individual components harmoniously balanced with one another. The properties of the individual constituents are employed in order to achieve an new overall image in the combination, wherein the characteristics of the ingredients retire into the background but without being suppressed.

In the context of the present invention, a “perfume oil” is a concentrated mixture of several fragrances which are employed, for example, in alcoholic solutions, for perfuming different products.

In the context of the present invention, a “scent theme” is the prevailing scent note in a fragrance composition.

In the context of the present invention, the “top note”, is the first phase of the scent progression of a perfume. It plays the decisive role during the first impression upon opening the bottle and while applying the perfume to the skin. The aim of the top note is to arouse interest in the perfume generally and to ensure attention. Consequently, an extraordinary character is often more important than a polished harmony. The top note is naturally determined by readily volatile fragrances.

In the context of the present invention, “modifying” signifies that the basic theme of a fragrance composition is provided with additional or different accords and odor nuances.

In the context of the present invention, “accords” are formed by combining different fragrances, which thus combine to give new odor images. The number of different fragrances used can range from two up to several hundred.

In the context of the present invention, an “organoleptically/sensorily effective amount” of a fragrance is the amount which suffices to produce a stimulatory effect on a sensory organ or sensory receptor.

(E/Z) represents (E and/or Z) and refers in principle, unless stated otherwise, not only to mixtures comprising both the stereoisomeric E configuration and the corresponding Z configuration, but also to the stereoisomerically pure E and Z forms of a compound.

### b) Special Embodiments of the Invention

The present invention relates especially to the following subjects:
1. A method for preparing cyclohexadec-8-en-1-one in stereoisomerically pure form or as a mixture of E and Z isomers, especially as a mixture of E and Z isomers, and cyclopentadecenone, particularly cyclopentadec-8-en-1-one and/or cyclopentadec-7-en-1-one, each in stereoisomerically pure form or as a mixture of E and Z isomers, especially as a mixture of E and Z isomers, comprising the following stages:
   i) reacting cyclohexadeca-1,9-diene, in stereoisomerically pure form or as a mixture of E and Z isomers (E/E, Z/Z, E/Z or Z/E), with N₂O to obtain a first reaction mixture (RM1) comprising cyclohexadec-8-en-1-one and cyclopentadecenyl carbaldehydes; in particular cyclopentadec-7/8-enyl-1-carbaldehydes, particularly cyclopentadec-8-enyl-1-carbaldehyde and cyclopentadec-7-enyl-1-carbaldehyde (each in stereoisomerically pure form or as a mixture of E and Z isomers),
   ii) reacting RM1 with oxygen in the presence of a Cu(II) catalyst, wherein the cyclopentadecenyl carbaldehydes present are converted by oxidative decarbonylation to one or more corresponding cyclopentadecenones, particularly cyclopentadec-8-en-1-one and cyclopentadec-7-en-1-one (each in stereoisomerically pure form or as a mixture of E and Z isomers), whereupon a second reaction mixture (RM2) is obtained; and
   iii) isolating (and in particular partially or completely separating) from RM2 the cyclohexadec-8-en-1-one and cyclopentadecenones formed, particularly the compounds of formulae I to VI specified below.
   The oxidative decarbonylation in stage ii) is preferably carried out in the presence of a catalytic amount of a preferably homogeneous Cu(II) catalyst and pure molecular oxygen, lean air or preferably air, particularly dried, filtered ambient air.
2. The method according to embodiment 1, wherein unreacted cyclohexadeca-1,9-diene is removed from RM1, preferably by distillation, whereupon a reaction mixture RM1a is obtained comprising cyclohexadec-8-en-1-one (e.g. in a proportion of at least 75%, for example, 75 to 85%) and cyclopentadecenyl carbaldehydes (e.g. in a proportion of at least 5%, for example, 5 to 10%), where RM1a in particular comprises less than 1% cyclohexadeca-1,9-diene; and wherein unreacted cyclohexadeca-1,9-diene is optionally fed back into the reaction after stage i).
   The % values in each case refer to the area % determined by GC analysis and correspond approximately to the % proportions by weight, based on the solids content of the mixture investigated.
3. The method according to embodiment 2, wherein RM1a is purified, preferably by distillation, whereupon a first fraction (F1) enriched in cyclohexadec-8-en-1-one is obtained particularly comprising less than 1%, for example less than 0.5 or 0.1% (e.g. 1.3-2.1 mmol) cyclopentadecenyl carbaldehydes, and at least one second fraction (F2) enriched in cyclopentadecenyl carbaldehydes is obtained, particularly comprising cyclohexadec-8-en-1-one (e.g. in a proportion of 20 to 80%, particularly approximately 30 to 60%, e.g. 50%) and at least 10%, particularly at least 20, preferably at least 30% or more, e.g. 30 to 70, or 35 to 60 or 40 to 50% (e.g. 30-100 g mixture per fraction) cyclopentadecenyl carbaldehydes.
   The % values in each case refer to the area % determined by GC analysis and correspond approximately to the % proportions by weight, based on the solids content of the mixture investigated.
4. The method according to any of the preceding embodiments, wherein RM1, RM1a or F2, in particular RM1a or F2, or a mixture thereof is used in stage ii).
5. The method according to any of the preceding embodiments, wherein a reaction mixture RM2a obtained from the oxidative decarbonylation (stage ii)) of fraction F2 is used in stage iii).
6. The method according to any of the preceding embodiments, wherein the conversion of the cyclopentadecenyl carbaldehydes in step ii) is at least 90%, e.g. 91 to 100% or 93 to 98%, based on the amount of carbaldehydes used.
7. The method according to any of the preceding embodiments, wherein cyclohexadec-8-en-1-one and cyclopentadecenones obtained from the oxidative decarbonylation in RM2 or RM2a are separated from each other by distillation.
8. The method according to any of the preceding embodiments, wherein the cyclohexadec-8-en-1-one is obtained in stage i) as a stereoisomeric mixture of (E)- and (Z)-cyclohexadec-8-en-1-one.
9. The method according to any of the preceding embodiments, wherein the cyclopentadecenyl carbaldehydes formed in stage i) are selected from (E/Z)-cyclopentadec-8-enyl-1-carbaldehyde, (E/Z)-cyclopentadec-7-enyl-1-carbaldehyde, individual stereoisomers thereof and mixtures of such stereoisomers.
10. The method according to any of the preceding embodiments, wherein the cyclopentadecenone formed in stage ii) is selected from (EIZ)-cyclopentadec-8-en-1-one, (E/Z)-cyclopentadec-7-en-1-one, individual stereoisomers thereof and mixtures of such stereoisomers (of the formulae I to IV).
11. The method according to any of the preceding embodiments, wherein the oxidative decarbonylation is carried out additionally in the presence of a base.
   The base used, for example, in the method according to the invention is, for example, selected from diazabicycloalkanes such as diazabicyclooctane (DABCO), diazabicycloundecene (DBU), diazabicyclononane (DBN) tertiary amines such as trimethylamine, triethylamine, triisopropylamine, diisopropylethylamine or tripropylamine, N,N-dimethylpiperazine, N-methylpyridine, N-methylpyrrolidone, quinuclidine and the like. Preference is given to using DBU.
12. The method according to any of the preceding embodiments, wherein the oxidative decarbonylation is carried out in the absence or in the presence of a solvent, e.g. in the presence of an organic solvent, in particular of an organic solvent, in which the catalyst is dissolved.
   Non-limiting examples of organic solvents include: polar, aprotic solvents such as dimethylformamide (DMF), hexamethylphosphoramide (HMPA), dimethyl sulfoxide (DMSO), tetramethylurea and dimethylacetamide, and also mixtures thereof. Further suitable organic solvents, which can be used alone or in combination with the above aprotic organic solvents, are alcanols such as methanol, ethanol, propanol or butanols such as tert-butanol and also tetrahydrofuran, dioxane or benzene. Preference is given to using DMF.
   In an alternative embodiment, the reaction is carried out essentially without addition of solvent, preferably in a solvent-free reaction mixture. The mixture of reactants and catalyst may preferably be heated for this purpose, for example to a temperature of 30 to 70° C. or 40 to 60° C.
13. The method according to any of the preceding embodiments, wherein the catalyst is formed in situ by adding a ligand to a Cu(II) salt, in particular a bidentate ligand, preferably a diamine ligand.
   The Cu(II) salt used according to the invention is selected, for example, from Cu(II) acetate, formate, sulfate, chloride or nitrate, preference being given to using Cu(OAc)₂. Suitable complexing ligands are particularly bidentate copper-complexing amine ligands such as N, N, N, N-tetramethylethylenediamine (TMEDA), 1,10-phenanthroline and 2,2-bipyridyl. Preference is given to using TMEDA.
14. The method according to any of the preceding embodiments, wherein the oxidative decarbonylation is carried out at a temperature in the range of 10 to 70, particularly 30 to 60° C., over a period of 0.1 to 40, particularly 1 to 30 h, preferably 2 to 7 h, above all 3 to 5 h, e.g. approximately 4 h, and at a pressure of 1 to 10, particularly 1 to 2 bar. It is also feasible to carry out the method in a vacuum, for example, at a reduced pressure in the range of 0.001 to 0.99 bar, or particularly 0.01 to 0.5 or 0.1 to 0.25 bar.
15. The method according to any of the preceding embodiments, wherein RM1, RM1a or F2 used in stage ii) comprises, in addition to (E/Z)-cyclohexadec-8-en-1-one, (E/Z)-cyclopentadec-8-enyl-1-carbaldehyde or a mixture of (E/Z)-cyclopentadec-8-enyl-1-carbaldehyde and (E/Z)-cyclopentadec-7-enyl-1-carbaldehyde or individual stereoisomers thereof.
16. The method according to any of the preceding embodiments, wherein the cyclopentadecenone obtained is at least one compound selected from (E/Z)-cyclopentadec-8-en-1-one and (E/Z)-cyclopentadec-7-en-1-one or individual stereoisomers thereof.
17. The method according to embodiment 16, wherein at least one compound is obtained selected from compounds of the following formulae I, II, III and IV

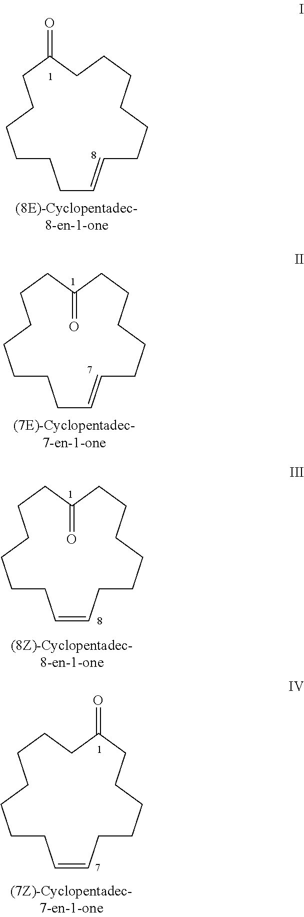 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

18. The method according to any of the preceding embodiments, wherein the isolation in stage iii) comprises a separation of RM2 by distillation and/or chromatography into at least one fraction F3 comprising cyclohexadec-8-en-1-one and at least one fraction F4 comprising at least one cyclopentadecenone (I-IV).
19. A substance or substance mixture obtainable by a method according to any of the preceding embodiments, wherein the substance or the substance mixture is selected from fractions F1, F3 and F4, or individual compounds isolated therefrom, optionally in stereoisomerically pure form; in particular substance mixture is selected from fractions F1, F3 and F4.
20. The use of at least one substance or substance mixture according to embodiment 19; in particular a substance mixture is selected from fractions F1, F3 and F4, as an aroma chemical, particularly as a fragrance.
21. The use according to embodiment 20 in agents selected from perfumes, washing and cleaning compositions, cosmetic agents, body care agents, hygiene articles, food, food supplements, scent dispensers, fragrances, pharmaceutical agents and plant protection agents.
22. The use according to either of embodiments 20 and 21 for generating a musk note in a fragrance composition.
23. The use according to embodiment 22, to impart, modify and/or enhance a musk scent note in a fragrance composition by admixing a sensorily effective amount of at least one substance or one substance mixture according to the definition in any of the embodiments 19.
24. A fragrance composition comprising at least one substance or one substance mixture according to the definition in claim 19, in particular a substance mixture is selected from fractions F1, F3 and F4, or is prepared according to one of the methods according to embodiments 1 to 18.
25. A composition according to embodiment 21, comprising the substance or the substance mixture in a proportion by weight of 0.01 to 99.9% by weight, 1 to 80% by weight, 2 to 50% by weight, 3 to 25 or 5 to 15% by weight, based on the total weight of the composition.
26. An agent comprising at least one substance or one substance mixture according to the definition in embodiment 19, in particular a substance mixture is selected from fractions F1, F3 and F4, or prepared according to one of the methods according to embodiments 1 to 18.
27. The agent according to embodiment 26, comprising the substance or the substance mixture in a proportion by weight of 0.01 to 99.9% by weight, 1 to 80% by weight, 2 to 50% by weight, 3 to 25 or 5 to 15% by weight, based on the total weight of the composition.
28. The agent according to embodiment 26 or 27 selected from perfumes, washing and cleaning compositions, cosmetic agents, body care agents, hygiene articles, food, food supplements, scent dispensers, fragrances, pharmaceutical agents and plant protection agents.
29. A method for purifying cyclohexadec-8-ene-1-one, wherein a mixture RM1a or F2 as defined in any of the method claims 1 to 3 is subjected to an oxidative decarbonylation as defined in any of the embodiments 1 or 11 to 14, and cyclohexadec-8-ene-1-one is separated from the oxidized decarbonylation products formed, particularly by distillation and/or chromatographically.

### c) Further Confugurations of the Invention

### c1) Fragrance Compositions:

According to a further aspect, “fragrances prepared according to the invention”, i.e. cyclohexadecenones and cyclopentadecenones, or mixtures thereof, as defined above, are also used, in stereoisomerically pure form or as a mixture of at least 2 stereoisomers, particularly for the purpose of efficient handling and dosing, as fragrance compositions with diluents or solvents. In this case, the proportion of fragrances, based on the sum total of fragrances and solvent, is given in % by weight.

### Solvent:

In the context of the present invention, a “solvent” serves as the diluent of the fragrances to be used according to the invention or the fragrance composition according to the invention but without having any odorous properties. Some solvents also have fixing properties.

The fragrances prepared according to the invention, a substance mixture defined above composed of several compounds/isomers thereof, can be added to 0.1 to 99% by weight of a diluent or solvent. Preference is given to at least 40% by weight solvents, more preferably at least 50% by weight solvents, further preferably at least 60% by weight solvents, more preferably at least 70% by weight solvents, particularly preferably at least 80% by weight solvents, especially preferably at least 90% by weight solvents, preferably in olfactory acceptable solvents.

Preferred olfactorily acceptable solvents are ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), benzyl benzoate (BB) and benzyl acetate. In this case, preference is given in turn to ethanol, diethyl phthalate, propylene glycol, dipropylene glycol, triethyl citrate, benzyl benzoate and isopropyl myristate.

In the context of the present invention, a “fragrance composition” is a mixture which, in addition to a “fragrance prepared according to the invention” or a substance mixture of two or more “fragrances prepared according to the invention” defined herein, optionally comprises at least one further fragrance. Such a fragrance composition may particularly take the form of a perfume composition (a perfume oil).

Fragrance compositions according to the invention comprise, for example, an amount of a “fragrance prepared according to the invention” or a substance mixture composed of two or more “fragrances prepared according to the invention” defined herein of 0.01 to 65% by weight, preferably of approximately 0.1 to approximately 50% by weight, preferably of approximately 0.5 to approximately 30% by weight and particularly preferably of approximately 0.5 to approximately 25% by weight, based on the total amount of the fragrance composition. The ratio by weight of (a) compound(s) prepared according to the invention to the total amount of further fragrances is in the range, for example, of 1:1000 to 1:0.5, preferably in the range of 1:700 to 1:1, particularly preferably in the range of 1:500 to 1:10.

Fragrance compositions according to the invention comprise, for example, an amount of “fragrance prepared according to the invention” or a substance mixture composed of two or more “fragrances prepared according to the invention” defined herein of 0.01 to 65% by weight and preferably of approximately 0.1 to approximately 50% by weight, preferably of approximately 0.5 to approximately 30% by weight and particularly preferably of approximately 0.5 to approximately 25% by weight, based on the total amount of the fragrance composition. The ratio by weight of compound(s) prepared according to the invention to the total amount of further fragrances (different therefrom) is in the range, for example, of 1:1000 to 1:0.5, preferably in the range of 1:700 to 1:1, particularly preferably in the range of 1:500 to 1:10.

The data above on the content of the compounds prepared according to the invention also apply correspondingly to the preferred compounds of the formulae I, II, III and IV, particularly also to the substance mixtures of the compounds of the formulae I, II, III and IV.

### Further Fragrances:

Fragrance compositions according to the invention, in addition to the “fragrance prepared according to the invention” or a substance mixture of two or more “fragrances prepared according to the invention” defined herein, additionally comprise at least one further fragrance, preferably 2, 3, 4, 5, 6, 7, 8 or more further fragrances, in which further fragrances are selected from, for example:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis-isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styralyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methytetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyl-oct-6-en-3-one (Claritone¹), 2,6-dimethyl-5-hepten-1-al (Melonal²), bomeol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), 7-methyl-2H-1,5-benzodioxepin-3(4H)-one (Calone1951⁵), 3,3,5-trimethylcyclohexyl acetate (preferably with a content of cis-isomers of 70% by weight) or more and 2,5,5-trimethyl-1,2,3,4,4a,5,6,7-octahydronaphthalen-2-ol (Ambrinol S¹). In the context of the present invention, the fragrances mentioned above are accordingly preferably combined with mixtures according to the invention.

If trade names are specified above, these refer to the following sources: ¹ Trade name of Symrise GmbH, Germany;² Trade name of Givaudan AG, Switzerland;³ Trade name of International Flavors & Fragrances Inc., USA;⁵ Trade name of Danisco Seillans S.A., France;⁹ Trade name of Firmenich S.A., Switzerland;¹⁰ Trade name of PFW Aroma Chemicals B.V., The Netherlands.

Further fragrances with which (E/Z)-cyclopentadec-7/8-en-1-one may be combined, for example, to give a fragrance composition are found, for example, in S. Arctander, Perfume and Flavor Chemicals, Vol. I and II, Montclair, N.J., 1969, Authors edition or K. Bauer, D. Garbe and H. Surburg, Common Fragrance and Flavor Materials, 4th. Ed., Wiley-VCH, Weinheim 2001. Specific examples are:

extracts from natural raw materials such as essential oils, concretes, absolutes, resins, resinoids, balsams, tinctures such as e.g.
ambergris tincture; amyris oil; angelica seed oil; angelica root oil; aniseed oil; valerian oil; basil oil; tree moss absolute; bay oil; lungwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; ylang ylang oil; hyssop oil; civet absolute; cinnamon leaf oil; cinnamon bark oil, and fractions thereof, or ingredients isolated therefrom;
individual fragrances from the group of hydrocarbons, such as e.g. 3 carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
the aliphatic alcohols such as e.g. hexanol; octanol; 3-octanol; 2,6-dimethylheptanol; 2 methyl-2-heptanol; 2-methyl-2-octanol; (E)-2-hexenol; (E) and (Z)-3-hexenol; 1 octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9 decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2 methylnonanal; (E)-2-hexenal; (Z)-4-heptenal; 2,6-dimethyl-5-heptenal; 10 undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-3 hexene; the aliphatic ketones and oximes thereof such as e.g. 2 heptanone; 2 octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;
the aliphatic sulfur-containing compounds such as e.g. 3-methylthiohexanol; 3 methylthiohexyl acetate; 3-mercaptohexanol; 3-mercaptohexyl acetate; 3-mercaptohexyl butyrate; 3-acetythiohexyl acetate; 1-menthene-8-thiol;
the aliphatic nitriles such as e.g. 2-nonenenitrile; 2-undecenenitrile; 2-tridecenenitrile; 3,12-tridecadienenitrile; 3,7-dimethyl-2,6-octadienenitrile; 3,7-dimethyl-6 octenenitrile;
the esters of aliphatic carboxylic acids such as e.g. (E) and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E,Z)-ethyl-2,4-decadienoate; methyl 2-octinate; methyl 2-noninate; allyl 2-isoamyloxy acetate; methyl-3,7-dimethyl-2,6 octadienoate; 4-methyl-2-pentyl crotonate;
the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatien-1-ol; and the formates, acetates, propionates, isobutyrates, butyrates, isovalerates, pentanoates, hexanoates, crotonates, tiglinates and 3-methyl-2 butenoates thereof;
the acyclic terpene aldehydes and ketones such as e.g. geranial; neral; citronellal; 7-hydroxy-3,7-dimethyloctanal; 7-methoxy-3,7-dimethyloctanal; 2,6,1-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpineol-4; menthan-8-ol; menthan-1-ol; menthan-7-ol; borneol; isobomeol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2 butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as e.g. alpha-3,3-trimethylcyclohexylmethanol; 1-(4 isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3-trimethyl-3-cyclopent-1-yl)pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1 (2,2,6 trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxymethoxy)cyclododecane; alpha-cedrene epoxide; 3a,6,6,9a tetramethyldodecahydronaphtho[2,1-b]furan; 3a-ethyl-6,6,9a-trimethyldodecahydronaphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1 ethoxyvinyl)-3,3,5,5-tetramethylcyclohexanone; 4-tert-pentylcyclohexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes such as e.g. 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones such as e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1 cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7 methanooctahydro-5 or 6-indenyl acetate;
the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexenecarboxylate; ethyl 2-methyl-1,3-dioxolane-2-acetate;
the araliphatic alcohols such as e.g. benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2 phenylethyl alcohol; 1,1-dimethyl-3-phenylpropanol; 1-ethyl-1-methyl-3-phenylpropanol; 2-methyl-5 phenylpentanol; 3-methyl-5-phenylpentanol; 3-phenyl-2-propen-1-ol; 4 methoxybenzyl alcohol; 1-(4-isopropylphenyl)ethanol;
the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;
the aromatic and araliphatic aldehydes such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone; 4-methylacetophenone; 4-methoxyacetophenone; 4-tert-butyl-2,6 dimethylacetophenone; 4-phenyl-2-butanone; 4-(4-hydroxyphenyl)-2-butanone; 1-(2 naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1 H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5-tert-butylbenzene; 3,5-dinitro-2,6-dimethyl-4-tert-butylacetophenone; cinnamonitrite; 3-methyl-5-phenyl-2-pentenonitrile; 3-methyl-5-phenylpentanonitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3 isopropylpyrazine; 2-isobutyl-3-methoxypyrazine;
the phenols, phenyl ethers and phenyl esters such as e.g. estragole; anethole; eugenol; eugenyl methyl ether isoeugenol; isoeugenyl methyl ether; thymol; carvacrol; diphenyl ether; beta-naphthyl methyl ether beta-naphthyl ethyl ether; beta-naphthyl isobutyl ether, 1,4-dimethoxybenzene; eugenyl acetate; 2-methoxy-4-methylphenol; 2-ethoxy-5-(1-propenyl)phenol; p-cresyl phenylacetate;
the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

### c2) Fragrance-Containing Articles

Fragrances prepared according to the invention or fragrance compositions according to the invention can be incorporated into a series of products or applied to said products.

Fragrances according to the invention can be used in the production of perfumed articles. The olfactory properties, like the material properties (such as solubility in customary solvents and compatibility with further customary constituents of such products), as well as the toxicological acceptability of the fragrances according to the invention underline their particular suitability for the stated use purposes. The positive properties contribute to the fact that the fragrances used according to the invention and the fragrance compositions according to the invention are particularly preferably used in perfume products, body care products, hygiene articles, textile detergents and in cleaners for solid surfaces.

The perfumed article is e.g. selected from perfume products, body care products, hygiene articles, textile detergents and cleaners for solid surfaces. Preferred perfumed articles according to the invention are also selected from among:

perfume products selected from perfume extracts, Eau de Parfums, Eau de Toilettes, Eau de Colognes, Eau de Solide, Extrait Parfum, air fresheners in liquid form, gel-like form or a form applied to a solid carrier, aerosol sprays, scented cleaners and scented oils;
body care products selected from aftershaves, pre-shave products, splash colognes, solid and liquid soaps, shower gels, shampoos, shaving soaps, saving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics such as e.g. eyeshadows, nail vamishes, make-ups, lipsticks, mascara, toothpaste, dental floss;
hygiene articles selected from candles, lamp oils, joss sticks, insecticides, repellents, propellants, rust removers, perfumed freshening wipes, armpit pads, baby diapers, sanitary towels, toilet paper, cosmetic wipes, pocket tissues, dishwasher deodorizer;
cleaners for solid surfaces selected from perfumed acidic, alkaline and neutral cleaners, such as e.g. floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

According to a further aspect, the fragrances used according to the invention and the fragrance compositions according to the invention are suitable for use in surfactant-containing perfumed articles. This is because fragrances and/or fragrance compositions with a rose top note and pronounced naturalness are often sought—especially for the perfuming of surfactant-containing formulations such as, for example, cleaners (in particular dishwashing compositions and all-purpose cleaners).

According to a further aspect, fragrances used according to the invention and fragrance compositions according to the invention can be used as agents for providing (a) hair or (b) textile fibers with a rosy odor note.

The fragrances to be used according to the invention and fragrance compositions according to the invention are therefore particularly well suited for use in surfactant-containing perfumed articles.

It is preferred if the perfumed article is one of the following:
an acidic, alkaline or neutral cleaner which is selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish, which is selected in particular from the group consisting of furniture polishes, floor waxes and shoe creams, or
a body care composition, which is selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

Ingredients with which fragrances used according to the invention or fragrance compositions according to the invention can preferably be combined are, for example: preservatives, abrasives, antiacne agents, agents to combat skin aging, antibacterial agents, anticellulite agents, antidandruff agents, anti-inflammatory agents, irritation-preventing agents, irritation-alleviating agents, antimicrobial agents, antioxidants, astringents, sweat-inhibiting agents, antiseptics, antistatics, binders, buffers, carrier materials, chelating agents, cell stimulants, cleaning agents, care agents, hair removal agents, surface-active substances, deodorizing agents, antiperspirants, emollients, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, detergents, fabric softeners, suspending agents, skin-tanning agents, thickeners, vitamins, oils, waxes, fats, phospholipids, saturated fatty acids, mono or polyunsaturated fatty acids, α-hydroxy acids, polyhydroxy fatty acids, liquefiers, dyes, color-protection agents, pigments, anticorrosives, aromas, flavorings, fragrances, polyols, surfactants, electrolytes, organic solvents or silicone derivatives.

According to a further aspect, the fragrances are used in the production of the perfumed articles in liquid form, undiluted or diluted with a solvent or in the form of a fragrance composition. Suitable solvents for this purpose are e.g. ethanol, isopropanol, diethylene glycol monoethyl ether, glycerol, propylene glycol, 1,2-butylene glycol, dipropylene glycol, diethyl phthalate, triethyl citrate, isopropyl myristate, etc. If the specified solvents have their own olfactory properties, they are assigned exclusively to the constituent “solvent” and not to the “fragrances”.

The fragrances and/or fragrance compositions present in the perfumed articles according to the invention can in this connection, in one embodiment, be absorbed onto a carrier, which ensures both fine distribution of the fragrance or fragrance composition within the product and also controlled release upon use. Carriers of this type may be porous inorganic materials such as light sulfate, silica gels, zeolites, gypsums, clays, clay granules, aerated concrete, etc. or organic materials such as woods and cellulose-based materials.

The fragrances used according to the invention and the fragrance compositions according to the invention can also be in microencapsulated form, spray-dried form, in the form of inclusion complexes or in the form of extrusion products and be added in this form to the product or article to be perfumed. The properties can be further optimized by so-called “coating” with suitable materials with regard to a more targeted release of the scent, for which purpose preferably waxy synthetic substances such as e.g. polyvinyl alcohol are used.

The microencapsulation can take place for example by the so-called coacervation method with the help of capsule materials, e.g. made of polyurethane-like substances or soft gelatin. The spray-dried perfume oils can be produced for example by spray-drying an emulsion or dispersion comprising the perfume oil, wherein carrier substances that can be used are modified starches, proteins, dextrin and vegetable gums. Inclusion complexes can be prepared e.g. by introducing dispersions of fragrance compositions and cyclodextrins or urea derivatives into a suitable solvent, e.g. water. Extrusion products can be prepared by melting fragrances used according to the invention and fragrance compositions according to the invention with a suitable wax-like substance and by extrusion with subsequent solidification, optionally in a suitable solvent, e.g. isopropanol.

### c3) Preparation of Fragrances According to the Invention

### i) NO₂O Oxidation According to Stage i)

The starting compound (which may be used either in stereoisomerically pure form or in the form of mixtures of stereoisomers) is the cyclic olefin cyclohexadeca-1,9-diene, which is either obtainable commerically or may be prepared according to Example 2 of WO 2012/084673.

In this particular case, a cyclic olefin is oxidized by reaction with dinitrogen monoxide. Dinitrogen monoxide may be used here in pure form or optionally diluted with other substances gaseous under the reaction conditions, such as carbon dioxide.

The reaction of the cyclic olefin with dinitrogen monoxide can be carried out without solvent or in the presence of at least one suitable solvent or diluent. The reaction is preferably carried out in the absence of solvent. All customary solvents and/or diluents are essentially suitable here, but with the proviso that they neither have a C—C double bond nor a C—C triple bond, nor an aldehyde group. Suitable solvents to be mentioned include, inter alia: cyclic alkanes, for example, cyclohexane, cyclopentane, cyclooctane, cyclododecane or saturated aliphatic or aromatic, optionally alkyl-substituted hydrocarbons.

The temperature in the reaction is, for example, from 140 to 350° C., in particular from 180 to 320° C. or from 200 to 300° C. It is also possible to carry out the reaction at two or more temperatures or in two or more temperature ranges which are in each case within the limits specified above. Temperature changes in the course of the reaction may be implemented continuously or discontinuously. However, the reaction temperature is essentially constant. However, the reaction may also be carried out adiabatically, such that the temperature increases in the reactor.

The pressure during the reaction of the cyclic olefin with dinitrogen monoxide is in particular higher than the autogenous pressure of the reactant or product mixture at the selected reaction temperature(s). The pressure is, for example, from 1 to 1000 bar, such as from 40 to 300 bar or from 50 to 200 bar.

It is possible to carry out the reaction of the cyclic olefin with dinitrogen monoxide at two or more pressures or in two or more pressure ranges which are in each case within the limits specified above. Pressure changes in the course of the reaction may be implemented continuously or discontinuously. However, the pressure during the reaction is essentially constant.

With regard to the reactors which can be used for the reaction (on a laboratory or production scale), there are no particular limitations. In particular, the reaction may be carried out in batch mode or in continuous mode. Consequently, the reactors used may be, for example, at least one CSTR (continuous stirred tank reactor) with at least one internal and/or at least one external heat exchanger, at least one tubular reactor, at least one tube bundle reactor or at least one loop reactor. It is also possible to configure at least one of these reactors such that it has at least two different zones. Such zones may differ in reaction conditions for example, such as the temperature or the pressure and/or in the geometry of the zone such as, for example, the volume or the cross section. If the reaction is carried out in two or more reactors, two or more identical reactor types or at least two different reactor types can be used. In particular, the reaction with dinitrogen monoxide is carried out in a single reactor. For example, the reaction may be carried out in batch mode or in continuous mode.

The residence time of the reaction mixture in the reactor is generally in the range from 0.1 to 40 hours, preferably in the range from 1 to 30 hours, more preferably in the range from 2 to 25 hours.

In the feed, the molar ratio of dinitrogen monoxide to the cyclic olefin is generally in the range from 0.01 to 30, for example in the range from 0.03 to 10, particularly preferably in the range from 0.05 to 1 and especially preferably in the range from 0.08 to 0.2.

Since dinitrogen monoxide is preferably used in deficiency, only a portion of the cyclohexadeca-1,9-diene is reacted. Unreacted cyclohexadeca-1,9-diene is separated from the reaction product by distillation and fed back again to the reaction. In this case, the unreacted cyclohexadeca-1,9-diene is the overhead product and the reaction product is the bottoms product of the column. The distillation in this case is conducted, for example, at a pressure at the top of 20 mbar and a bottom temperature of 210° C. The differential pressure across the column is around 18 mbar. The column may be equipped with a structured fabric packing of the Montz A3 type. The packing height is, for example, 4 m and the feed is, for example, 2 m.

From the bottoms output obtained, the cyclopentadecenyl carbaldehyde formed as secondary component can be isolated (enriched) by distillation and may be further purified by chromatography. In particular, however, the crude product mixture of cyclohexadec-8-en-1-one and carbaldehydes thus obtained is used directly in stage (ii).

Therefore, the direct reaction of the reaction mixture (RM1) is preferably carried out in stage (ii) without any further purification.

In a further preferred variant, a further purification by distillation is carried out to obtain reaction mixture RM1a.

In the case of an additional purification, this can be carried out, for example, by distillation (such as in particular by fractional distillation, preferably under reduced pressure) or chromatography, the purification preferably being carried out by distillation. Suitable purification methods are familiar to those skilled in the art. The purification may be carried out, for example, in batch mode or continuously.

For example, the distillation by means of a distillation column may use packings known to those skilled in the art. The optimal distillation conditions can be established by those skilled in the art without undue effort. The distillation can be carried out in particular under vacuum, for example at a pressure of <1000 mbar, <500 mbar, <300 mbar, <100 mbar or <10 mbar. The distillation column used may have several, for example, at least 20, at least 25 or at least 30, such as up to 70 theoretical plates. The reflux ratio can be, for example, in the range of about 5 to 100 and be at least 20, at least 25 or at least 30 and is in particular about 100 for a particularly advantageous fractionation.

Column chromatography can also be carried out, for example, in place of or in addition to purification by distillation. In this case, column materials and mobile phases known to those skilled in the art are used. Optimal chromatography conditions, such as column geometry and mobile phase flow rate, can be established by those skilled in the art without undue effort.

Examples of suitable column materials are polar adsorbents such as iron oxide Fe₂O₃, aluminum oxide, carbohydrates or silica gel, with or without additives such as fluorescence indicators or gypsum.

Examples of suitable mobile phases are: aliphatic or aromatic mobile phases such as alkanes or cycloalkanes, for example pentane, petroleum ether, hexane, heptane, toluene or the corresponding cyclic compounds; aliphatic ethers, esters or, for example, Et₂O, MTBE, EtOAc, acetone or mixtures of such mobile phases such as hexane/MTBE, hexane/EtOAc, pentane/Et₂O, petroleum ether/Et₂O.

In this case, one or more fractions (F2) can be isolated with an increased carbaldehyde content having a carbaldehyde content of more than 20, for example, more than 30, more than 40, more than 50, more than 60, more than 70 or more than 80%. The % values in each case refer to the area % determined by GC analysis and correspond approximately to the % proportions by weight, based on the solids content of the mixture investigated.

In addition, one or more fractions (F1) can be isolated enriched in cyclohexadec-8-en-1-one (content approximately 98%), and in particular having less than 1% cyclopentadecenyl carbaldehyde. The % values in each case refer to the area % determined by GC analysis and correspond approximately to the % proportions by weight, based on the solids content of the mixture investigated.

The carbaldehydes and cyclohexadec-8-en-1-one may be isolated here in stereoisomerically pure form, or in particular as a mixture of two or more stereoisomers.

### ii) Oxidative Decarbonylation According to Stage ii)

Starting from the reaction product from stage i) (i.e. RM1, RM1a or F2), the oxidative decarbonylation is carried out according to Cu(II)-based decarbonylations known from the prior art, cf. e.g. a) Tetrahedron Letters 1969, 12, 985; U.S. Pat. No. 3,496,197; b) Tetrahedron Letters 1995, 4641, c) Org. Lett. 2010, 2630, d) Bioorg. Med. Chem. Lett. 2013, 23, 5949, e) Chin. Chem. Lett. 2014, 25, 771.)

The catalytic reaction of the cyclic carbaldehydes with molecular oxygen according to the invention takes place in the presence of at least one suitable solvent or diluent. The reaction may also be carried out without a solvent.

In this case, oxygen can be used in the process as pure oxygen or preferably as a constituent of ambient air or lean air. By way of example, the reaction may be carried out with airflows in the range of 0.5-100 Nl/h, preferably about 50 Nl/h. The air may be passed into the reaction mixture by means of a frit for example. Alternatively, the gas may also be passed into the reaction by means of a tube or a nozzle.

Examples of suitable solvents include, inter alia: polar, aprotic solvents such as dimethylformamide (DMF), hexamethylphosphoramide (HMPA), dimethyl sulfoxide (DMSO), tetramethylurea and dimethylacetamide, and also mixtures thereof. Further suitable organic solvents, which can be used alone or in combination with the above aprotic organic solvents, are alcanols such as methanol, ethanol, propanol, isopropanol or butanols such as tert-butanol and also tetrahydrofuran, dioxane or benzene.

Cu(II)-based catalysts, particularly homogeneous catalysts, are used as catalyst. These are preferably formed in situ in the reaction mixture by adding in particular a bidentate ligand, preferably a diamine ligand, to a Cu(II) salt.

The Cu(II) salt used according to the invention is selected, for example, from Cu(II) acetate, formate, sulfate, chloride or nitrate, preference being given to using Cu(OAc)₂.

Suitable complexing ligands are particularly bidentate copper-complexing amine ligands such as N, N, N′, N′-tetramethylethylenediamine (TMEDA), 1,10-phenanthroline and 2,2′-bipyridine. Preference is given to using TMEDA.

Complex ligand and Cu(II) salt are used in an approximately equimolar ratio. The molar proportion of complex is approximately 0.1 to 10, particularly 1 to 5, preferably approximately 2.5 mol %, based on the carbaldehyde used.

In particular, the decarbonylation is carried out, in addition, in the presence of an organic base. The base used, for example, in the method according to the invention is selected from diazabicycloalkanes such as diazabicyclooctane (DABCO), diazabicycloundecene (DBU), diazabicyclononane (DBN) tertiary amines such as trimethylamine, triethylamine, diisopropylethylamine or tripropylamine, N,N-dimethylpiperazine, N-methylpyridine, N-methylpyrrolidone, quinuclidine and the like. Preference is given to using DBU. The base is used here in a proportion of 0.1-1 equivalents, 0.2-0.8 equivalents or particularly preferably 0.4-0.6 equivalents, based on the carbaldehydes used.

The temperature in the reaction is, depending on reactants and solvent used, for example, from 20 to 100° C., such as in particular from 30 to 80° C., particularly from 40 to 60° C. It is also possible to carry out the reaction at two or more temperatures or in two or more temperature ranges which are in each case within the limits specified above. Temperature changes in the course of the reaction may be implemented continuously or discontinuously. However, the reaction temperature is essentially constant.

The pressure during the reaction of the carbaldehydes with oxygen is particularly ambient pressure or approximately in the range of the autogenous pressure of the reactant or product mixture at the selected reaction temperature(s). The pressure is, for example, from 1 to 5 bar, such as from 1 to 3 bar, preferably about 1 bar.

It is possible to carry out the reaction at two or more pressures or in two or more pressure ranges which are in each case within the limits specified above. Pressure changes in the course of the reaction may be implemented continuously or discontinuously. In particular, the pressure during the reaction is essentially constant.

In particular, the reaction may be carried out in batch mode or in continuous mode.

With regard to the reaction vessels which can be used for the reaction (on a laboratory or production scale), there are no particular limitations. In the case of using a reactor, customary stirred reactors, CSTR (continuous stirred tank reactor), in each case with or without internal and/or external heat exchangers, a tubular reactor, a tube bundle reactor or a loop reactor may be used. It is also possible to configure the reactor such that it has at least two different zones. Such zones may differ in reaction conditions for example, such as the temperature or the pressure and/or in the geometry of the zone such as, for example, the volume or the cross section. If the reaction is carried out in two or more reactors, two or more identical reactor types or at least two different reactor types can be used. In particular, however, the reaction is carried out in a single reactor, particularly a stirred reactor.

The residence time of the reaction mixture in the reactor is generally in the range from 0.1 to 40 hours, preferably in the range from 5 to 30 hours, more preferably in the range from 2 to 7 hours.

In particular, the reaction can be carried out as follows:

A reaction mixture (RM1, RM1a) consisting of an excess of (E/Z)-cyclohexadec-8-en-1-one and an amount of cyclopentadec-7/8-enyl carbaldehyde present in deficit, or a reaction mixture (F2) consisting of an excess of cyclopentadec-7/8-enyl carbaldehyde and an amount of (E/Z)-cyclohexadec-8-en-1-one present in deficit, is dissolved in DMF. To the mixture is added as base diazabicycloundecene (DBU, 0.5 to 1.5, e.g. 0.6 eq based on carbaldehyde). A stream of air is passed continuously through the mixture. The bidentate complex ligand TMEDA (1 to 5 mol %, e.g. 2 mol % based on carbaldehyde) and Cu(OAc)₂ (1 to 5 mol %, 2 mol %) are dissolved in DMF. The Cu-TMEDA mixture is then added dropwise continuously to the reaction solution or added directly (in one portion). The reaction was stirred at 40 to 60, e.g. 50° C. for 3 to 30 h, e.g. 5 h. EtOAc and water is then added. The aqueous phase is adjusted to pH 4 by means of 98% H₂SO₄. The organic phase is extracted. The aqueous phase is then optionally washed again with EtOAc. The combined organic phases are dried, filtered and concentrated under vacuum.

The residue is then optionally further processed. For example, a fractional distillation is carried out. The cyclopentadecenones according to the invention can thus be separated from unreacted cyclohexadec-8-en-1-one. The cyclopentadecenones can then optionally be separated from low boilers, which were not removed by distillation, by column chromatography. For this purpose, for example, silica gel as stationary phase and, as mobile phase, a mixture of cyclohexane:EtOAc, for example by elution using a stepwise gradient 100:1/30:1/20:1, are suitable. The product is eluted at around 80:1.

Alternatively, column chromatography can be carried out in place of the fractional distillation. The mixture consisting of cyclopentadecenones and cyclohexadecenones can be purified in this case by column chromatography using a mobile phase of cyclohexane:EtOAc (30:1→20:1). (Column material: silica gel F₂₅₄)

The invention is elucidated in detail with reference to the non-limiting working examples below:

### Experimental Section

### Methods:

### Gas Chromatography (GC)

Separating column: CP-Wax 52CB 25 m×0.32 mm×1.2 μm 1 ml/min N₂
Conditions: 90°-5 min-10°/min-240°-30 min Inj/Det 200°/250° (Method A)
Conditions: 80-3°/min-250°-Inj/Det 200°/250° (Method B) (Example 2 only)
Sample volumes: 0.2 ml

### GC/MS

Separating column: CP-Wax 52 CB (1.2 μm film thickness), split ratio 10:1
Conditions: 80°-3 min-240°-30 min 0.2 μl
MS conditions: 25-785 amu, 70 eV

### GC/IR

Detector MCT/A wavelength 650-4000 cm⁻¹
Cells/transfer line temperature 250° C.

### Scan 6

### Resolution 8

### Column Chromatography

A glass column with frit base was used. The column was packed to ⅔ with swollen silica gel F₂₅₄. The solvent mixture was forced through the column using a positive pressure of 0.2 to 0.4 bar.

### Example 1: Reaction of cyclohexadeca-1,9-diene (CHDD) with NO₂O

In an adiabatic tubular reactor (12 m length, 10 cm diameter), 2000 g/h of CHDD were reacted with 40 g/h of N₂O at a reactor inlet temperature of 210° C. and 100 bar. The conversion of CHDD was 12%. Unreacted CHDD was removed by distillation (20 mbar, 190° C. bottom temperature) from the product mixture comprising Globanone® and cyclopentadecenyl carbaldehydes I-IV and fed back to the system.

Otherwise the reaction was conducted as described in PCT/EP2015/072544 or the applicant of WO 2012/084673.

### Comparative Example 1: Distillation of the Output from Example 1

The reaction output from Example 1 was fractionally distilled under vacuum (<10 mbar) and a bottom temperature of 200° C. in a column having theoretical plate number of 70 and a reflux ratio of 100. The cyclohexadec-8-enone thus prepared still comprises undesired cyclopentadecenyl carbaldehydes.

The distillate was assessed olfactorily: plastic note, aldehydic, musk

### Example 2: Cu-Catalyzed Oxidative Decarbonylation of the Product from Example 1

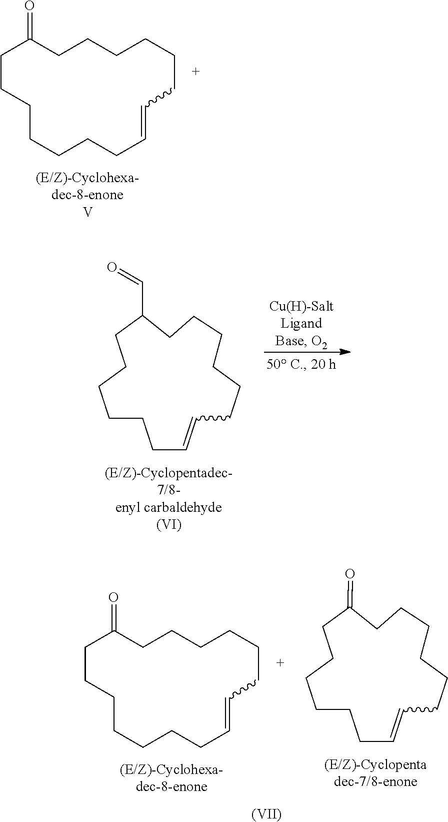 [SMILES: O=C1CCCCCCC=CCCCCCCC1 ; O=CC1CCCCCCC=CCCCCCC1 ; O=C1CCCCCCC=CCCCCCCC1 ; O=C1CCCCCCC=CCCCCCC1 ; "(VII)"]

100 g of a mixture consisting of 81% (E/Z)-cyclohexadec-8-enone (V) and 5.9% cyclopentadec-7/8-enyl carbaldehyde (VI) were dissolved in 300 ml of DMF. To the mixture, 2.3 g of diazabicycloundecene (DBU, 15.2 mmol, 0.6 eq) were added. A stream of air was passed continuously through the mixture. TMEDA (120 mg, 0.5 mmol, 2 mol %) and Cu(OAc)₂ (90 mg, 0.5 mmol, 2 mol %) were dissolved in 30 ml of DMF. The Cu-TMEDA mixture was added dropwise to the reaction mixture continuously over 8 h via a syringe pump. The reaction was stirred at 50° C. for 20 h. 300 ml of EtOAc were then added and 200 ml of 0.1 M HCl. The organic phase was extracted. The aqueous phase was then washed once again with EtOAc (2×100 ml). The combined organic phases were dried over Na₂SO₄, filtered and concentrated under vacuum. The conversion of the carbaldehydes was >95%. The selectivity of the cyclopentadecenones was 95%. Cyclohexadec-8-enone was not affected.

The residue was worked up by means of fractional distillation in a spinning band column with 20 theoretical plates at 1 mbar top pressure and 125-129° C. head temperature and 170-180° C. bottom temperature. The reflux ratio was 75.

The cyclopentadec-7/8-enones were separated from cyclohexadec-8-enone (yield 85%).

The cyclopentadec-7/8-enones were subsequently separated from low boilers, which could not be removed in the distillation, by column chromatography (silica gel, mobile phase cyclohexane:EtOAc 100:1, 30:1, 20:1), such that 1 g of a colorless oil was obtained with a purity of 84% (3.7 mmol).

Proportion: (E)-cyclopentadec-8-enone=54%
(Z)-cyclopentadec-8-enone=30%
(E)-cyclopentadec-7-enone=0.45%

¹H NMR (500 MHz, CDCl₃, 25′C): s=5.3 (m, 2H), 2.5-2.3 (m, 4H), 2.1-1.9 (m, 4H), 1.7-1.55 (m, 4H), 1.45-1.10 (m, 12H). (of the mixture)

trans-cyclopentadec-8-enone I

¹³C-NMR (125 MHz, CDCl₃, 25° C.): σ=211.98 (C═O), 130.99 (C═C), 41.49 (2×CH₂), 31.80 (2×CH₂), 28.26 (2×CH₂), 28.21 (2×CH₂), 26.84 (2×CH₂), 26.83 (2×CH₂), 22.91 (2×CH₂).

cis-cyclopentadec-8-enone III

¹³C-NMR (125 MHz, CDCl₃, 25° C.): σ=211.93 (C═O), 130.44 (C—C), 41.57 (2×CH₂), 31.80 (2×CH₂), 28.48 (2×CH₂), 27.68 (2×CH₂), 25.74 (2×CH₂), 23.16 (2×CH₂).

MS m/z=222, 204, 179, 165, 152, 135, 125, 111, 98, 81, 67, 55, 41.

trans-cyclopentadec-8-enone I

IR (ATR) [symbol] [cm⁻¹]=3022, 2934, 2864, 1724, 1449, 1356, 1121, 969.

cis-cyclopentadec-8-enone III

IR (ATR) [symbol] [cm⁻¹]=3011, 2935, 2866, 1723, 1456, 1354, 716.

In the GC, trans-cyclopentadec-7-enone II could also be detected in the mixture at a fraction of 0.45%. However, insufficient material was available for a spectroscopic evaluation of the compound. The compound could be identified by GC/MS-IR

trans-cyclopentadec-7-enone II

IR (ATR) [symbol] [cm⁻¹]=3020, 2934, 2864, 1723, 1449, 1353, 1121, 969.

Compound IV could also be identified from a further reaction batch by GC/MS-IR:

cis-cyclopentadec-7-enone IV

IR (ATR) [symbol] [cm⁻¹]=3012, 2936, 2866, 1723, 1457, 1353, 714.

Smell test (mixture of I-III, 84%): green, aldehydic, harsh, gassy, musk-like (84% cyclopentadec-8-enone)

### Example 3: Fine Distillation of the Output from Example 1 to Obtain a First Cut (F1) Composed of Cyclohexadec-8-Enone with Very Little Cyclopentadecenyl Carbaldehydes and a Second Cut (F2) Composed of Cyclohexadec-8-Enone with a High Proportion of Cyclopentadecenyl Carbaldehydes

2600 g of a mixture from Example 1 (crude output from oxidation of cyclohexadeca-1,9-diene with N₂O) having in total ca. 5% cyclopentadecenyl carbaldehyde were fractionally distilled in a batch column (Sulzer fabric packing DX, separation height 2000 mm, diameter 43 mm, top pressure: 5 mbar, pressure loss across the column: 5 mbar, bottom temperature: 180° C., Sambay evaporator, reflux ratio: 100). Mixtures of various proportions of cyclopentadec-7/8-enyl carbaldehyde and cyclohexadec-8-enone were obtained in this case in different fractions. The respective content (area %, corresponding approximately to % by weight) was determined by gas chromatography.

In particular, the following were obtained:

Cut F1: Proportion of cyclohexadec-8-enone 98%; proportion of cyclopentadec-7/8-enyl carbaldehyde 1%.

Cut F2: Cyclopentadec-7/8-enyl carbaldehyde (50%)

### Example 4: Cu-Catalyzed Oxidative Decarbonylation of the F2 Cut Having a High Proportion of Cyclopentadecenyl Carbaldehydes

10 g of a mixture F2 from Example 3 essentially consisting of 30% cyclohexadec-8-enone (V) and 50% cyclopentadecenyl carbaldehyde (VI) were dissolved in 30 ml of DMF. To the mixture, 1.93 g of DBU (12.7 mmol, 0.6 eq) were added. A stream of air was passed continuously through the mixture. 2,2′-Bypyridine (0.06 g, 0.4 mmol, 2 mol %) and Cu(OAc)₂ (0.08 g, 0.4 mmol, 2 mol %) were dissolved in 5 ml of DMF. The Cu-bipy mixture was added dropwise to the reaction solution continuously over 8 h via a syringe pump. The reaction was stirred at 50° C. for 20 h. 100 ml of EtOAc were then added and 100 ml of 0.1 M HCl. The organic phase was extracted. The aqueous phase was then washed once again with EtOAc (2×50 ml). The combined organic phases were dried over Na₂SO₄, filtered and concentrated under vacuum. The residue was investigated by gas chromatography. The conversion of carbaldehyde was >95%, the selectivity to cyclopentadecenone was 95%. The conversion of cyclohexadec-8-enone was 0%.

The mixture consisting of cyclopentadecenone and cyclohexadecenone was purified by column chromatography using a mobile phase of cyclohexane:EtOAc (30:1→20:1). (Column material: silica gel F₂₅₄). 1.8 g of a pale yellowish oil were obtained comprising 49% cyclopentadecenone and 43% cyclohexadecenone.

Smell test: weakly of musk, metallic, aldehydic, plastic note

### Example 5: Cu-Catalyzed Oxidative Decarbonylation without Solvent

18.5 g of diazabicycloundecene (DBU, 121.8 mmol, 0.6 eq), 0.6 g of TMEDA (5.1 mmol, 2.5 mol %) and 0.92 g of Cu(OAc)₂ (5.1 mmol, 2.5 mol %) were added to 400 g of a mixture consisting of 81% (E/Z)-cyclohexadec-8-enone (V) and 5.9% cyclopentadec-8-enyl carbaldehyde (VI). The reaction mixture was heated to 50′C and an airflow (100 Nl/h) was passed continuously through the mixture. The mixture was stirred for 4 h. 200 ml of EtOAc, 200 ml of water and 43.4 g of AcOH were then added at room temperature and the mixture was stirred for 5 min. The phases were separated and the aqueous phase was re-extracted with 200 ml of EtOAc. The combined organic phases were dried over Na₂SO₄, filtered and concentrated. The conversion of the carbaldehydes was 97%. The selectivity for cyclopentadecenones was 85%. The loss of cyclohexadec-8-enone was 0.2%.

All % figures are based on % by weight.

The disclosure of the publications mentioned herein is explicitly incorporated by reference.

1.-17. (canceled)

18. A method for preparing cyclohexadec-8-en-1-one and cyclopentadecenone comprising the following stages:
i) reacting cyclohexadeca-1,9-diene with N₂O to obtain a first reaction mixture (RM1) comprising cyclohexadec-8-en-1-one and cyclopentadecenyl carbaldehydes;
ii) reacting RM1 with oxygen in the presence of a Cu(II) catalyst, wherein the cyclopentadecenyl carbaldehydes present are converted by oxidative decarbonylation to cyclopentadecenones, whereupon a second reaction mixture (RM2) is obtained; and
iii) isolating cyclohexadec-8-en-1-one and cyclopentadecenone from RM2.

19. The method according to claim 18, wherein unreacted cyclohexadeca-1,9-diene is removed from RM1, whereupon a reaction mixture RM1a is obtained comprising cyclohexadec-8-en-1-one and cyclopentadecenyl carbaldehydes, where RM1a comprises less than 1% cyclohexadeca-1,9-diene; and wherein unreacted cyclohexadeca-1,9-diene is optionally fed back into the reaction after stage i).

20. The method according to claim 19, wherein RM1a is purified, whereupon a first fraction (F1) enriched in cyclohexadec-8-en-1-one is obtained comprising less than 1% cyclopentadecenyl carbaldehydes, and at least one second fraction (F2) enriched in cyclopentadecenyl carbaldehydes is obtained comprising cyclohexadec-8-en-1-one with more than 10% cyclopentadecenyl carbaldehydes.

21. The method according to claim 20, wherein RM1, RM1a or F2, or a mixture thereof is used in stage ii).

22. The method according to claim 20, wherein a reaction mixture RM2a obtained from the oxidative decarbonylation (stage ii)) of fraction F2 is used in stage iii).

23. The method according to claim 22, wherein cyclohexadec-8-en-1-one and cyclopentadecenones obtained from the oxidative decarbonylation in RM2 or RM2a are separated from each other by distillation.

24. The method according to claim 18, wherein the cyclopentadecenone formed in stage ii) is selected from the group consisting of (E/Z)-cyclopentadec-8-en-1-one, (E/Z)-cyclopentadec-7-en-1-one, individual stereoisomers thereof and mixtures of such stereoisomers.

25. The method according to claim 18, wherein the Cu(II) catalyst in stage ii) is formed in situ by adding a ligand to a Cu(II) salt.

26. The method according to claim 20, wherein RM1, RM1a or F2 used in stage ii) comprises, in addition to (E/Z)-cyclohexadec-8-en-1-one, (E/Z)-cyclopentadec-8-enyl-1-carbaldehyde or a mixture of (E/Z)-cyclopentadec-8-enyl-1-carbaldehyde and (E/Z)-cyclopentadec-7-enyl-1-carbaldehyde.

27. The method according to claim 18, wherein the cyclopentadecenone obtained is at least one compound selected from the group consisting of (E/Z)-cyclopentadec-8-en-1-one and (E/Z)-cyclopentadec-7-en-1-one.

28. The method according to claim 27, wherein at least one compound is obtained selected from the group consisting of compounds of formulae I, II, III and IV

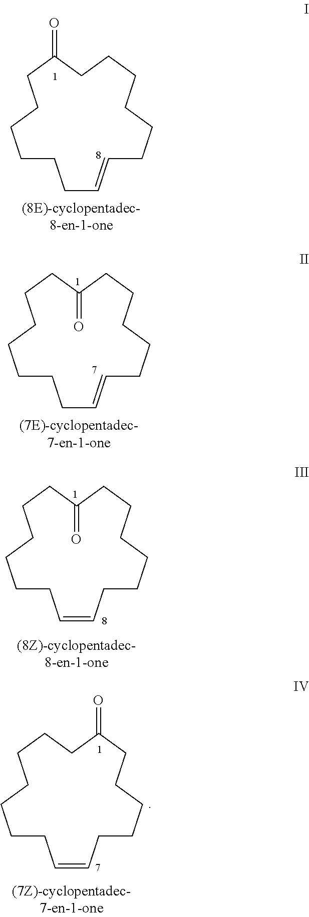 [SMILES: O=C1CCCCCC/C=C/CCCCCC1 ; O=C1CCCCC/C=C/CCCCCCC1 ; O=C1CCCCCC/C=C\CCCCCC1 ; O=C1CCCCC/C=C\CCCCCCC1]

29. The method according to claim 18, wherein the isolation in stage iii) comprises a separation of RM2 by distillation and/or chromatography into at least one fraction F3 comprising cyclohexadec-8-en-1-one and at least one fraction F4 comprising at least one cyclopentadecenone (I-IV).

30. A substance or substance mixture obtained by the method according to claim 18, wherein the substance or the substance mixture is selected from fractions F1, F3 and F4, or individual compounds isolated therefrom, optionally in stereoisomerically pure form.

31. A composition comprising at least one substance or substance mixture according to claim 30 as an aroma chemical.

32. A fragrance composition comprising at least one substance or one substance mixture prepared according the method according to claim 18.

33. An agent comprising at least one substance or one substance mixture according to the definition in claim 30.

34. A method for purifying cyclohexadec-8-en-1-one, wherein a mixture RM1 or RM1a or F2 as defined in the method claim 20 is subjected to an oxidative decarbonylation, and cyclohexadec-8-en-1-one is separated from the oxidized decarbonylation products formed, by distillation and/or chromatographically.
